(19) United States
(12) Patent Application Publication
(10) Pub. No.: US 2005/0021127 A1
(43) Pub. Date: Jan. 27, 2005

(54) Porous glass fused onto stent for drug retention

(76) Inventor: Paul John Kawula, Sunnyvale, CA (US)

Correspondence Address: Charles E. Runyan;Squire, Sanders & Dempsey L.L.P., Suite 300, One Maritime Plaza, San Francisco, CA, 94111, US

(21) Appl. No.: 10/623,908

(22) Filed: Jul. 21, 2003

(51) Int. Cl. A61F 2/06

(52) U.S. Cl. 623/1.15; 427/2.25; 623/1.42

(57) ABSTRACT

An implantable medical device that comprises an attached porous ceramic component is disclosed. This ceramic component joins the surface of the medical device to an auxiliary component such as a glass, plastic, ceramic, or metal device. This auxiliary component can be fiber optic, can be an electrode, or can be a sensor or chip-based sensor. Alternatively, the ceramic component is a drug reservoir capable of locally delivering a drug or other therapeutic substance near the implantation site of the medical device.

### FIELD OF THE INVENTION

This invention relates to medical devices comprising glass frit material that binds other components to the medical device or that stores one or more drugs for local delivery within a body lumen.

### DESCRIPTION OF THE BACKGROUND

Blood vessel occlusions are commonly treated by mechanically enhancing blood flow in the affected vessels, such as by employing a stent. Stents act as scaffolding, functioning to physically hold open and, if desired, expand the wall of the vessel (or passageway, if used in other body lumens). Typically, stents are compressible for insertion through small lumens using catheters. Once they are at the desired location they are expanded to a larger diameter. U.S. Pat. No. 4,733,665 issued to Palmaz, U.S. Pat. No. 4,800,882 issued to Gianturco, and U.S. Pat. No. 4,886,062 issued to Wiktor all disclose stents.

Stents are used not only for mechanical intervention, but also as vehicles for providing biological therapy by medicating the stents. Such stents locally administer a therapeutic substance. Local delivery is useful because the medication is concentrated at a specific site, and smaller medication amounts can be administered than with systemic dosing. Systemic dosing often produces adverse or even toxic side effects.

Using a polymeric carrier coated onto the stent surface is one method of medicating a stent. A composition including a solvent, a dissolved polymer, and a dispersed therapeutic substance is used for applying the coating. Immersing the stent in the composition or spraying the stent with the composition yields the desired coating. After the solvent evaporates, the stent surfaces have a polymer coating that contains the therapeutic substance.

A shortcoming of using a polymeric carrier is that, as the drug mass increases, the polymer mass typically must also increase, maintaining a defined ratio between the two. This increases the volume of material deposited onto the stent. Depending on the shape of the stent, this increase can cause polymer cracking in subsequent processing steps. In addition to mechanical problems, excess material could have other biological effects that offset the benefits of using drug-containing stents. Therefore, ways of increasing drug mass without a corresponding increase of the polymer binder are desired.

### SUMMARY

The present invention is directed to a medical device for implanting in a patient. It features a ceramic component disposed on its surface. In some embodiments, this component comprises a porous region and, optionally, a second less porous region. In some embodiments, at least one attachment region is disposed in or on the surface. In some embodiments, an oxide layer is disposed within or on the attachment region, between the surface of the medical device and the ceramic component or the ceramic component's less porous region.

In some embodiments, the ceramic component releasably contains a drug; in other embodiments it serves as an attachment point for another component, e.g. fiber optics, sensors, electrodes, etc. Methods for making invention devices are also within the scope of the invention.

### BRIEF DESCRIPTION OF THE FIGURES

FIG. 1 illustrates a portion of an implantable medical device.

FIG. 2 illustrates a portion of an implantable medical device in cross-section.

FIG. 3 illustrates a ceramic component adapted for attachment to an implantable medical device.

FIG. 4 illustrates an implantable medical device with an attached ceramic component.

FIG. 5 illustrates an implantable medical device with attached ceramic component and polymeric overlayer.

FIG. 6 illustrates an implantable medical device connected to an auxiliary component through a ceramic component with a cross-section taken through the ceramic component.

FIG. 7 illustrates an implantable medical device with an oxide layer, in cross-section.

FIG. 8 illustrates an implantable medical device with an oxide layer, in cross-section.

FIG. 9 illustrates a prototypical medical device surface and a cross-section of that device.

FIG. 10 illustrates a prototypical medical device surface and a cross-section of that device.

FIG. 11 illustrates a prototypical device surface prepared using a laser and a cross-section of that surface.

### DETAILED DESCRIPTION

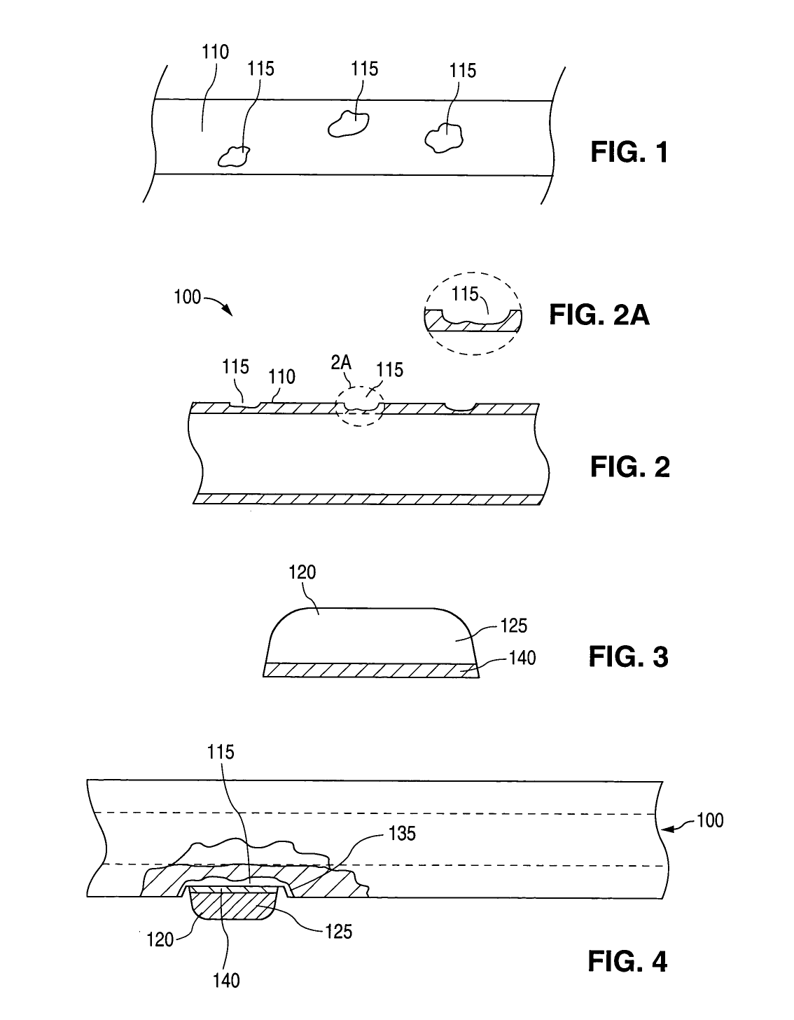

As can be seen by reference to FIGS. 1 and 2, the implantable medical device 100 comprises a surface 110 with at least one attachment region 115 to which a glass or ceramic component 120 attaches. (FIG. 3) The term “ceramic component” encompasses components comprising ceramic or glass unless otherwise indicated.

When the ceramic component 120 attaches to the surface 110, the attachment region 115 is a portion of the surface 110. Alternatively, an attachment region is formed in the surface 110 by removing material. In those cases, the attachment region 115 is the surface left behind after the material has been removed. Attachment regions 115 are formed in any material to which the ceramic component 120 can attach using one of the attachment methods described below. In some embodiments, attachment regions 115 are formed in surfaces 110 that are metal. In some of these embodiments, the metal is selected from stainless steel, tantalum, niobium, super-elastic nickel-titanium alloys, titanium, silver, gold, platinum, steel, aluminum, or any other material that produces an oxide layer that the glass frit will wet and adhere to. In other embodiments, an attachment region 115 is formed in polymeric material. As can be seen by reference to FIG. 1, the surface 110 has one or more attachment regions 115. The number and the geometry of attachment regions 115 are determined by the implantable medical device's intended function, by the composition of the surface 110, and by the composition of the ceramic component 120.

In addition to implantable medical devices comprising metal surfaces, some inventive embodiments comprise devices with polymeric surfaces 110 or attachment regions 115. These polymeric surfaces 110 or attachment regions 115 are based on organic or inorganic polymers. For purposes of this disclosure, organic polymers are polymers formed from monomers containing a carbon-based backbone. Specific examples of organic polymers include ethylene vinyl alcohol copolymer (commonly known by the generic name EVOH or by the trade name EVAL); poly(hydroxyvalerate); poly(L-lactic acid); polycaprolactone; poly(lactide-co-glycolide); poly(hydroxybutyrate); poly(hydroxybutyrate-co-valerate); polydioxanone; polyorthoester; polyanhydride; poly(glycolic acid); poly(D,L-lactic acid); poly(glycolic acid-co-trimethylene carbonate); polyphosphoester; polyphosphoester urethane; poly(amino acids); cyanoacrylates; poly(trimethylene carbonate); poly(iminocarbonate); copoly(ether-esters); polyalkylene oxalates; polyphosphazenes; fibrin; fibrinogen; cellulose; starch; collagen; hyaluronic acid; polyurethanes; silicones; polyesters; polyolefins; polyisobutylene and ethylene-alphaolefin copolymers; acrylic polymers and copolymers; vinyl halide polymers and copolymers; polyvinyl chloride; polyvinyl ethers; polyvinyl methyl ether; polyvinylidene halides; polyvinylidene fluoride polyvinylidene chloride; polyacrylonitrile; polyvinyl ketones; polyvinyl aromatics; polystyrene; polyvinyl esters; polyvinyl acetate; copolymers of vinyl monomers with each other and olefins; ethylene-methyl methacrylate copolymers; acrylonitrile-styrene copolymers; ABS resins; ethylene-vinyl acetate copolymers; polyamides; Nylon 66; polycaprolactam; alkyd resins; polycarbonates; polyoxymethylenes; polyimides; polyethers; epoxy resins; polyurethanes; rayon; rayon-triacetate; cellulose; cellulose acetate; cellulose butyrate; cellulose acetate butyrate; cellophane; cellulose nitrate; cellulose propionate; cellulose ethers; or carboxymethyl cellulose. For purposes of this disclosure, inorganic polymers are polymers formed from monomers containing a backbone with elements primarily selected from those other than carbon or hydrogen.

The ceramic component 120 is shown in FIG. 3. In some embodiments, it comprises a porous area 125. In some of these embodiments, it also comprises a nonporous or less porous vitrified area 140 adjacent to the attachment region 115. This area is also called a fusing layer. In some embodiments, the fusing layer 140 is formed when the ceramic component 120 is fused to the attachment region 115.

Some inventive embodiments comprise devices with glass or ceramic surfaces 110. For purposes of this disclosure, “ceramic” takes its standard meaning. The term encompasses materials that either have or lack long-range order. Connections between metallic elements and nonmetallic elements typically predominate in a ceramic. For purposes of this disclosure, “glass” is defined as a material that is a solid at room temperature, but that lacks long-range order. Invention-suitable glasses are organic or inorganic. Inorganic glasses typically comprise connections between oxygen and silicon or aluminum and are a subset of ceramics. Organic glasses typically comprise carbon and hydrogen connections. In some embodiments the ceramic component comprises a material that has both ceramic and glass characteristics. In some embodiments the glass is chosen to have an coefficient of expansion within 15% of the coefficient of expansion of the surface of the medical device.

Specific examples of glasses useful in practicing this invention include borosilicate glass, lead glass, soda glass, uranium glass, soft glass, fused quartz, and fused silica. Specific examples of ceramics useful in practicing this invention include carbide ceramics, oxide ceramics, nitride ceramics, and boride ceramics. Specific examples of these include titania, zirconia, hafnia, silica, alumina, silica alumina, silicon carbide, tungsten carbide, silicon boronitride, boronitride, silicon, or gallium arsenide.

In some embodiments, the ceramic component is made from a glass frit material. Glass frit is calcined or partly fused material, but is usually porous and not yet vitrified. For purposes of this disclosure, glass frit takes its normal definition as is known to those of ordinary skill in the medical device art and as is known to those of ordinary skill in the glass making art.

As can be seen from reference to FIG. 4, a bonding or fusing layer 140 is disposed between the attachment region 115, and the ceramic component 120, in some embodiments. This fusing layer 140 connects to the attachment region 115 on one side and to the ceramic component 120 on the other. In some embodiments, the fusing layer 140 is integral to the ceramic component 120. An oxide layer 135 can be disposed between the attachment region 115 and the fusing layer 140. This oxide layer 135 improves the connection between the attachment region 115 and the fusing layer 140.

In general, the ceramic components can be used in at least two ways. In some embodiments, the ceramic component is used as is. In some of these embodiments, the porous region of the ceramic component serves as a reservoir for drugs or other therapeutically active substances that are to be administered, locally or otherwise, inside the patient. In such cases, the ceramic component's porosity may be controlled during the manufacturing steps (discussed below) so that the available volume within the pores of the ceramic component(s) is large enough to contain the desired amount of drug. Porosity control also allows drug-delivery-rate control. One of ordinary skill in the art recognizes that smaller channels between the pores and the surface of the ceramic component will tend to slow drug delivery.

In inventive embodiments where the ceramic component releasable contains a drug, the ceramic component is sometimes called a drug reservoir. “Drug reservoir” is used to refer to an individual reservoir or to refer to all of the individual reservoirs as a collection. The drug comprises any biologically active material that can be loaded into the drug reservoir using the methods described below and that can diffuse or otherwise exit from the drug reservoir after medical-device implantation. Suitable drugs comprise active agents.

The active agent can be for inhibiting the activity of vascular smooth muscle cells. More specifically, the active agent can be aimed at inhibiting abnormal or inappropriate migration or proliferation of smooth muscle cells to prevent, inhibit, reduce, or treat restenosis. The active agent may be any substance capable of exerting a therapeutic or prophylactic effect in the practice of the present invention. Examples of such active agents include antiproliferative, antineoplastic, antiinflammatory, antiplatelet, anticoagulant, antifibrin, antithrombin, antimitotic, antibiotic, and antioxidant substances as well as combinations thereof. An example of an antiproliferative substance is actinomycin D, or derivatives and analogs thereof (manufactured by Sigma-Aldrich 1001 West Saint Paul Avenue, Milwaukee, Wis. 53233; or COSMEGEN available from Merck). Synonyms of actinomycin D include dactinomycin, actinomycin IV, actinomycin I₁, actinomycin X₁, and actinomycin C₁. Examples of antineoplastics include paclitaxel and docetaxel. Examples of antiplatelets, anticoagulants, antifibrins, and antithrombins include aspirin, sodium heparin, low molecular weight heparin, hirudin, argatroban, forskolin, vapiprost, prostacyclin and prostacyclin analogs, dextran, D-phe-pro-arg-chloromethylketone (synthetic antithrombin), dipyridamole, glycoprotein IIb/IIIa platelet membrane receptor antagonist, recombinant hirudin, thrombin inhibitor (available from Biogen), and 7E-3B® (an antiplatelet drug from Centocor). Examples of antimitotic agents include methotrexate, azathioprine, vincristine, vinblastine, fluorouracil, adriamycin, and mutamycin. Examples of cytostatic or antiproliferative agents include angiopeptin (a somatostatin analog from Ibsen), angiotensin converting enzyme inhibitors such as CAPTOPRIL (available from Squibb), CILAZAPRIL (available from Hoffman-LaRoche), or LISINOPRIL (available from Merck & Co., Whitehouse Station, N.J.), calcium channel blockers (such as Nifedipine), colchicine, fibroblast growth factor (FGF) antagonists, histamine antagonist, LOVASTATIN (an inhibitor of HMG-CoA reductase, a cholesterol lowering drug from Merck &Co.), monoclonal antibodies (such as PDGF receptors), nitroprusside, phosphodiesterase inhibitors, prostaglandin inhibitor (available form Glazo), Seramin (a PDGF antagonist), serotonin blockers, thioprotease inhibitors, triazolopyrimidine (a PDGF antagonist), and nitric oxide. Other therapeutic substances or agents that may be appropriate include alpha-interferon, genetically engineered epithelial cells, dexamethasone, estradiol, clobetasol propionate, cisplatin, insulin sensitizers, receptor tyrosine kinase inhibitors and carboplatin. Exposure of the composition to the active agent should not adversely alter the active agent's composition or characteristic. Accordingly, the particular active agent is selected for compatibility with any other components of the drug.

Rapamycin is an exemplary active agent. Additionally, 40-O-(2-hydroxy)ethyl-rapamycin, or a functional analog or structural derivative thereof, are also exemplary active agents. Examples of analogs or derivatives of 40-O-(2-hydroxy)ethyl-rapamycin include but are not limited to 40-O-(3-hydroxy)propyl-rapamycin and 40-O-[2-(2-hydroxy)ethoxy]ethyl-rapamycin.

As illustrated in FIG. 5, the medical device can optionally comprise a polymeric overlayer 250. This polymeric layer 250 affects the drug's loading into the reservoir or its diffusion from the reservoir. It may also mechanically or chemically protect the drug reservoir. Representative examples of polymers useful as layers over the drug reservoir or medical device include ethylene vinyl alcohol copolymer, poly(hydroxyvalerate); poly(L-lactic acid); polycaprolactone; poly(lactide-co-glycolide); poly(hydroxybutyrate); poly(hydroxybutyrate-co-valerate); polydioxanone; polyorthoester; polyanhydride; poly(glycolic acid); poly(D,L-lactic acid); poly(glycolic acid-co-trimethylene carbonate); polyphosphoester; polyphosphoester urethane; poly(amino acids); cyanoacrylates; poly(trimethylene carbonate); poly(iminocarbonate); copoly(ether-esters) (e.g. PEO/PLA); polyalkylene oxalates; polyphosphazenes; biomolecules, such as fibrin, fibrinogen, cellulose, starch, collagen and hyaluronic acid; polyurethanes; silicones; polyesters; polyolefins; polyisobutylene and ethylene-alphaolefin copolymers; acrylic polymers and copolymers; vinyl halide polymers and copolymers, such as polyvinyl chloride; polyvinyl ethers, such as polyvinyl methyl ether; polyvinylidene halides, such as polyvinylidene fluoride and polyvinylidene chloride; polyacrylonitrile; polyvinyl ketones; polyvinyl aromatics, such as polystyrene; polyvinyl esters, such as polyvinyl acetate; copolymers of vinyl monomers with each other and olefins, such as ethylene-methyl methacrylate copolymers, acrylonitrile-styrene copolymers, ABS resins, and ethylene-vinyl acetate copolymers; polyamides, such as Nylon 66 and polycaprolactam; alkyd resins; polycarbonates; polyoxymethylenes; polyimides; polyethers; epoxy resins; polyurethanes; rayon; rayon-triacetate; cellulose; cellulose acetate; cellulose butyrate; cellulose acetate butyrate; cellophane; cellulose nitrate; cellulose propionate; cellulose ethers; and carboxymethyl cellulose.

A second use of invention ceramic components is as attachment means for at least one auxiliary component 200. The auxiliary component 200 comprises glass, ceramic, metallic, plastic, or polymeric portions. For instance, a fiber-optic strand or fiber can serve as the auxiliary component 200. In that case, the ceramic component 120 connects the fiber-optic fiber to the surface 110 or into the attachment region 115. Alternatively, the auxiliary component is a chip-based device, e.g. a sensor such as a physical sensor (which measures temperature, pressure, etc.) or a chemical sensor (which measures pH, drug concentration, etc.). Such an assembly allows the sensor to contact body fluids or tissues very near the medical device's implantation site.

When the auxiliary component 200 comprises metal or a metal device, the ceramic component 120 connects the metal of the auxiliary component 200 to the surface 110. By varying the ceramic component's composition or geometry, this connection can be made insulating or conductive. Therefore, for some embodiments a metal electrode serves as the auxiliary component 200 and attaches to the metallic surface 120 of the medical device, with the ceramic component insulating it from the metallic surface 120.

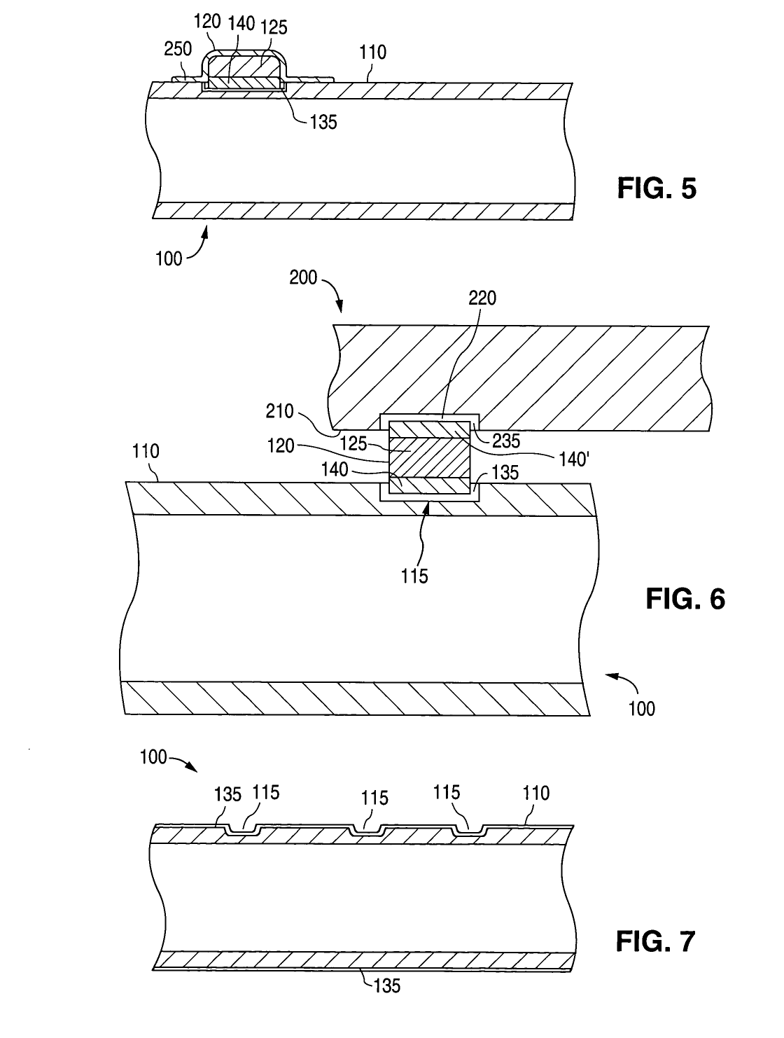

When the auxiliary component 200 comprises glass or ceramic, the ceramic component 120 connects the glass or ceramic of the auxiliary component 200 to the surface 110. Thus, in some embodiments a glass or ceramic auxiliary component 200 is adhered to an implantable medical device's metallic surface 110 (for those devices with a metallic surface) or to some other surface material of an implantable medical device. In inventive embodiments in which the ceramic component 120 acts as an attachment means, the ceramic component's thickness varies to suit the particular application. FIG. 6 shows an implantable medical device 100 comprising a metallic surface 110 attached to an auxiliary component 200 comprising a metal electrode. The auxiliary component 200 has a surface 210 and attachment region(s) called auxiliary-surface attachment regions 220. Both metallic surfaces 110 & 210 have attachment regions 115 & 220 lined with an oxide layer 135 & 235. The ceramic component 120 is disposed between these oxide layers. In this type of embodiment, the ceramic component has two fusing layers 140 & 140′, each one to mate with the oxide layers 135 & 235, respectively. The relative thickness of the two fusing layers 140 & 140′ and the porous layer 125 depends upon the intended application. In some embodiments, the entire porous layer 125 becomes vitrified. In some of these embodiments, the fusing layer 140 is indistinguishable from the porous layer 125. While in others, the ceramic component retains a distinguishable porous layer 125.

Ultimately, invention medical devices typically comprise at least one connection between dissimilar materials. For instance, if the surface 110 is metallic and the surface of the ceramic component 120 is glasseous, the connection is between metal and glass. When making such a connection, those of ordinary skill in the art desire that the dissimilar materials nonetheless be compatible with each other and have similar thermal characteristics to each other, such as similar coefficients of thermal expansion. Similar thermal characteristics usually ensure that the temperature changes in the connected pieces will cause similar size changes in the pieces. Any unmatched size change across a connection creates mechanical strain across the connection—the larger the mismatch, the greater the strain.

Unfortunately, matching thermal characteristics is usually insufficient to allow materials as dissimilar as metal and glass to be connected, especially when the pieces to be connected have the geometries envisioned for invention ceramic-metal or ceramic-polymer connections. This means that the materials and the connection must be able to withstand the mechanical stress described above. The particular device, such as the strut of a stent, can be adjusted or machined to make its thermal expansion more like the ceramic's, and the ceramic composition can be selected to make its thermal expansion more like the strut's. But whether this matching occurs or not, the connection should be strong enough to substantially overcome the mechanical strain introduced by mismatches between the connected objects' thermal characteristics.

Connection strength partially depends on the compatibility between the materials actually present at the interface between the objects. In some embodiments, an oxide layer is formed on the surface of the metal. At positions in this oxide layer near the metal, the oxide layer's composition is similar to that of the metal beneath it; while at the oxide surface, the composition is very much like that of glass or ceramic. Thus, the layer serves as a transition between the metal and glass or other auxiliary component substance.

The ceramic component may also serve as a transition layer between the surface 110 and an auxiliary component 200. Because thermal expansion parameters are important, dissimilarity in thermal expansion between two materials is enough for the materials to be considered dissimilar for purposes of this invention. A glass medical device bonded to a glass auxiliary component could result in a connection between dissimilar materials, depending upon each component's related thermal parameters. Thus, some inventive embodiments comprise a glass surface of a medical device connected to a glass surface of the auxiliary component connected through the ceramic component 120.

In a typical embodiment, a machining means is used to manufacture the surface 110 to contain attachment regions 115. Examples of well-known machining means include grinding, eroding, stamping, forging, molding, casting, cutting, etc. These are accomplished conventionally or using lasers. Some embodiments use laser ablation to form the attachment regions 115. Electrical discharge machining, ultrasonic machining, sputter machining and electropolishing can also serve as the machining means in this invention.

After the attachment regions 115 are formed, their surfaces should be processed to receive the ceramic component 120. The processing steps depend on which materials compose the region surrounding the attachment regions 115. When the region is metallic, two different processes are employed to treat the attachment region surfaces. For some metals the surface of the metal can be directly attached to the ceramic component 120 either because the surface 115 is compatible with the ceramic component material or because the surface 115 naturally has an appropriate oxide layer 135 that is compatible with the ceramic component material.

For other metals, such as stainless steel, an oxide layer 135 is formed on the attachment region 115 surfaces. This is accomplished by heating just the attachment region 115 using a localized heating means, such as directing a laser at the surfaces or using some other localized thermal processing means as are widely employed in the art. Alternatively, the entire surface is heated. With this method, areas with undesired oxide may in some cases have the oxide removed before further processing. FIG. 7 shows an implantable medical device 100 with attachment regions 115 machined into its surface 110 after the surface 110 has been heated to form an oxide layer 135. Other methods of forming oxide layers on surfaces are known to those of ordinary skill in the art and are considered to be within the scope of this disclosure. Other useful heating means include lasers, hydrogen furnaces, high-voltage DC arc current, etc. One of ordinary skill in the art is versed in suitable heating methods.

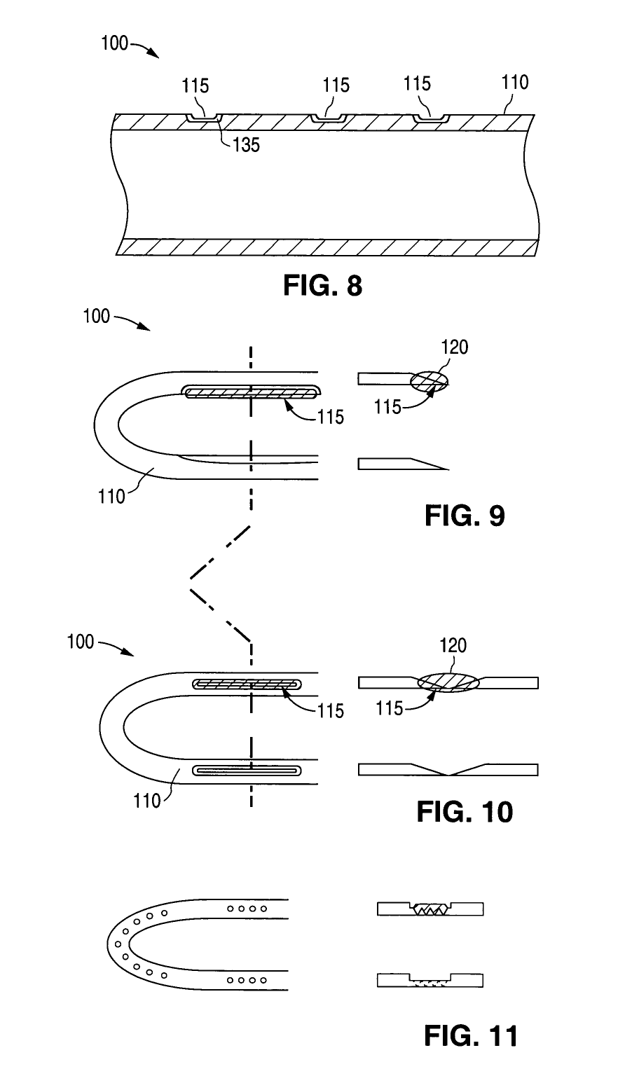

FIG. 8 shows the implantable medical device of FIG. 7 after the oxide layer has been removed from undesired areas. Note that oxide layer 135 remains on the surfaces of the attachment regions 115.

In alternative embodiments, the oxide layer 135 is formed by masking the surface 110 in those areas where an oxide layer is undesired. Then the device is locally heated or heated in its entirety, as described above. Alternatively, a material that promotes surface oxidation or that oxidizes the surface is selectively applied to areas where the oxide layer is desired. Then, if necessary, the device is locally heated or heated in its entirety. Masking techniques are well known in the art. Silk screening and transfer tape methods can be used in the practice of this invention.

Once the oxide layer 135 has been formed on the attachment regions 115, the ceramic component 120 is applied. Application occurs by forming the ceramic component material in situ or pre-forming it and placing it within or on the attachment region 115.

In some embodiments, forming the ceramic component in situ is accomplished by applying a precursor material that can be chemically transformed into a glass or ceramic material. One suitable precursor comprises a material that is known in the glassmaking art as glass frit. Glass frit is a partially calcined, porous material. In some embodiments, the precursor is a slurry comprising the glass frit and binders. The binders allow the precursor to be laid down like paint. Thus, the precursor sticks to the surface until the processing steps described below are completed. In some embodiments, precursor materials are gels or hydrogels, as is known in the art. Upon further treatment these gels form porous, networked structures.

The precursor materials are applied using conventional means such as dipping, spraying, painting, depositing using chemical vapor deposition, or otherwise applying the composition to the medical device. The precursor materials are applied to the entire device or selectively applied directly to the attachment regions. In some embodiments, areas of the medical device not requiring the ceramic component 120 are masked to prevent adhesion of the precursor materials. In some embodiments, precursor material is applied directly to the attachment regions using a device comprising a needle applicator and a pump.

Once the precursor material has been applied as required, it is converted to the ceramic component 120. This conversion has several steps. In typical embodiments, the precursor material is heated to remove any binders, leaving behind the glass frit material or the networked structure from the gel. Therefore, the temperature of the precursor material should be placed within a range to remove the binders from the material at a reasonable rate without introducing unwanted changes or disruptions into the glass frit or the networked structure. This step is accomplished by heating the entire medical device or by locally heating the precursor material such as with a laser or other local heating technique. Next, the ceramic component 120 is fused to the oxide layer 135 previously deposited in the attachment region 115, or, for surfaces not requiring an oxide layer, fused directly to the attachment region 115 itself. In typical embodiments, fusing is accomplished with a heating step, as well. Usually fusing requires higher, but more localized temperatures. Again, the heating is accomplished by heating the entire device or by local heating. The goal here is to heat the oxide layer 135 and the adjacent area of the glass frit material so that these regions fuse. In some embodiments, heating is carried out so that the bulk of the porous region 125 remains substantially unaltered, i.e. remains porous. Thus, local heating, confined to the oxide-layer-glass-component junction, such as with a laser, is frequently selected.

This heating step creates the fusing layer 140 described above. With sufficient heat, the glass-frit material vitrifies. Since in some embodiments this step is carried out in the presence of the oxide layer 135 and in some embodiments the oxide layer 135 is substantially compatible with the glass frit material, the materials fuse (in regions where high enough temperature is maintained for long enough times). For purposes of this invention, compatibility between the oxide layer and the ceramic component or glass frit material means that they fuse or sinter together when heated. Heating parameters should be chosen so that fusion occurs. Those of ordinary skill in the art know how to determine these parameters. The two heating steps described here need not be practiced separately. In other words, the heating step that removes the binder may also be the step that fuses the ceramic component 120 to the oxide layer 135 and creates the fusing layer 140.

As discussed above, glass-frit material is a calcined glass-precursor material. As such, if heated to high enough temperature it will vitrify and rapidly become non-porous. This means that, for applications in which it is not desired to change the porosity of the frit, the heating should be controlled such that the slurry binders are driven off, but that the frit does not substantially vitrify or substantially lose its porosity. During any fusing steps, the heating should also be controlled to substantially retain frit porosity. This leaves a material with bulk porosity similar to that of the frit originally found in the slurry. Some embodiments warrant a different glass porosity from that of the frit originally found in the slurry. In those cases, the heating may include an annealing step at a temperature below the melting point of the frit. Those of ordinary skill in the art know how to anneal.

Porosity for gels is similar to that for the glass-frit material. Typically, the method of preparing the gel and subsequent heat treatment determines the porosity of the resulting network. This network can also be subject to porosity modification using annealing steps, as discussed above.

In some embodiments, the ceramic component 120 can also be applied by first pre-forming a ceramic component. The component 120 is shaped and sized to match the medical device attachment regions 115 and is temporarily joined to attachment region 115 either by physically holding it in place or by applying a temporary adhesive. Then, as described above, the ceramic component 120 is fused to the attachment-region surfaces. The vitrified fusing layer 140 can be pre-formed in the ceramic component 120 before it is attached to the attachment region 115 or it can be formed during the fusing step. Ceramic component porosity is selected according to the intended use.

After the ceramic component 120 has been attached to the surface 110, it can be machined to change its shape, as necessary. For instance, some embodiments machine the ceramic component 120 so that its outer surface is substantially coplanar with the surface 110. Such machining methods are well known to those of ordinary skill in the art.

After the ceramic component 120 has been attached to the surface 110, it can be used as a drug reservoir or used to attach an auxiliary component 200.

When the ceramic component 120 is to serve as a drug reservoir, further manufacturing steps are necessary. First and foremost, a drug is deposited in the drug reservoir. Suitable drugs are described above. It is well within the skill level of one of ordinary skill in the art to prepare suitable drugs or compositions comprising suitable drugs. Once prepared, the drug is loaded into the drug reservoir 120 by spraying, painting, etc., the material onto the drug reservoir 120 or by dipping the drug reservoir 120 into the material. After the drug reservoir 120 is filled, any excess drug is removed using methods known to those of ordinary skill in the art.

Those of ordinary skill in the art are well versed in methods of coating medical devices with polymeric layers such as layer 250. The medical device of the instant invention can comprise a polymeric layer 250 over the drug reservoir 120 or over portions of the medical device. The polymeric layer 250 can be laid down before the drug reservoir 120 is filled, if the layer is porous to the drug or composition. The polymeric layer 250 can also be laid down after the reservoir is filled. For those embodiments that comprise a polymeric layer 250 covering the drug reservoir 120, the layer's composition and structure should be chosen so that the drugs can diffuse from the drug reservoir 120 through the polymeric layer 250 to the treatment site inside the patient. With appropriate selection, the polymeric layer 250 can further control the drug delivery rate.

Those of ordinary skill in the art may readily envision other ways of controlling the drug delivery rate from invention drug reservoirs; these are considered to be within the scope of the invention.

When the ceramic component 120 is used as an attachment means, further manufacturing steps are necessary, as well. Primarily, the surface of the auxiliary component 210 must be prepared to contain the necessary auxiliary-component attachment regions 220 and oxide layer 235 as was described above for the surface 115. As above, the ceramic component 120 can be prepared in-situ or pre-formed. The ceramic component 120 is attached to the attachment regions of both the implantable medical device and the auxiliary component. This is done as described above, except that at least two connections are formed: at least one between the ceramic component 120 and attachment region 115; and at least one between the ceramic component 120 and the auxiliary-component attachment region 220. These connections can be created sequentially, as described above for a single connection, or can be created substantially simultaneously.

When these connections are created substantially simultaneously, a method of holding the medical device 100, the ceramic component 120, and the auxiliary component 200 together and in registry with each other should be used until the connections are made. This is accomplished with a temporary adhesive, with an appropriate mechanical clamp, or using any other method as is known to those of ordinary skill in the art.

In some embodiments, the connections are created as described above using local heating, such as with a laser. In other embodiments, the entire structure including the medical device 100, ceramic component 120, and auxiliary component 200 is heated. Various combinations of locally heating one connection while heating the entire portion of another, if such complications are necessary for the device's intended function, are within the scope of this invention.

One of ordinary skill in the art recognizes that more than one auxiliary component 200 can similarly function in the practice of this invention. For example, invention medical devices 100 can be constructed with an auxiliary component 200, such as a chemical sensor, and a separate auxiliary component 200, such as an electrode.

In some embodiments, the ceramic component 120, when used as an attachment means, is constructed to retain the porosity necessary to allow its simultaneous use as a drug reservoir, as described above.

Those of ordinary skill in the art are well versed in methods of coating medical devices with polymeric layers. Some invention medical devices comprise a polymeric layer 250 over the auxiliary components 200 or over larger portions of the medical device 100.

The above discussion relates to fusing metal substrates to glass or ceramic substrates. One of ordinary skill will recognize that the technique described above also function with plastic or other polymeric substrates. In most of these cases, the plastic substrate is connected directly to the ceramic component without an intervening oxide layer. Also, the amount of heat required for the connection between the ceramic component and the plastic surface of the medical device or auxiliary component is typically much less than that required for the surfaces discussed above, as in known to those of ordinary skill in the art. In some cases, this lower amount of heat does not cause a second less porous region to form in the ceramic component. Therefore, in those cases, the embodiments may lack a second less porous region. In some cases, use of a suitable plastic allows for the connection between the plastic surface and the porous ceramic component to be made using an adhesive. Thus, a porous glass drug reservoir can be attached to a polymeric implantable medical device.

The surface 110 of the implantable medical device 100 can be machined so that its thermal characteristics are transformed such that after machining they match the thermal characteristics of the ceramic component more closely.

One way of accomplishing this is to machine a knife-edge or feathered edge 1115 into the surface 110 within the attachment region 115. This method is similar to a standard house keeper seal typically used in glass-to-metal seals.

Another way of accomplishing this is shown in FIG. 11. This figure shows a prototypical medical device 100 where the surface 110 has been machined. A CO₂ laser drilled-out attachement regions 115 in the surface 110. This method is well within the skill level of those of ordinary skill in the art. Typically, the laser will drill out a crater 1135 and then the laser will be repositioned and another crater 1135 will be drilled out. The process is repeated until a large enough attachment region 115 has been machined into the surface 110.

This process results is many craters 1135 that are very close to each other. The craters 1135 are close enough together that their walls overlap forming a psuedo-feathered edge reminiscent of the edge in a seal made using the house keeper technique.

As one of ordinary skill in the art will recognize, either of these techniques can be readily adapted to the embodiments described in this document.

The following disclose representative embodiments. These are by way of example only and should not be construed as the only embodiments.

In one set of inventive embodiments, the medical device attaches to the ceramic component through an oxide layer formed on the surface of attachment regions machined into the medical device's surface. The ceramic component has a less porous region at or near where it attaches to the oxide layer and a porous region substantially throughout the remainder. This porous region is filled with a drug at some time before use. In some of these embodiments, the surface of the medical device is a metal that comprises iron, cobalt, nickel, manganese, stainless steel, tantalum, niobium, super-elastic nickel-titanium alloys, titanium, silver, gold, platinum, steel, or aluminum. In some of these embodiments, attachment uses a laser as a heat source. In some of these embodiments, the ceramic component forms on heating the glass-frit slurry.

In another set of inventive embodiments, the medical device attaches to the ceramic component through an oxide layer formed on the surface of attachment regions machined into the medical device's surface. The ceramic component has a second less porous region at or near where it attaches to the oxide layer and a porous region substantially throughout the remainder. This porous region is filled with a drug at some time before use. In some of these embodiments, the surface of the medical device is a metal such as stainless steel, tantalum, super-elastic nickel-titanium alloys, titanium, silver, gold, platinum, or steel. In some of these embodiments, the medical device is an inter-vascular stent. In some of these embodiments, attachment uses a laser as a heat source. In some of these embodiments, the ceramic component forms on heating the glass-frit slurry.

In another set of inventive embodiments, the medical device attaches to the ceramic component through an oxide layer formed on the surface of attachment regions machined into the medical device's surface. The ceramic component has a second less porous region at or near where it attaches to the oxide layer and a porous region substantially throughout the remainder. This porous region is filled with a drug at some time before use. In some of these embodiments, the surface of the medical device is a metal that comprises iron, cobalt, nickel, manganese, stainless steel, tantalum, niobium, super-elastic nickel-titanium alloys, titanium, silver, gold, platinum, steel, or aluminum. In some of these embodiments, the drug contains a smooth-muscle-cell vascular activity inhibitor, a wound healing enhancer, an agent for improving the structural properties in a vascular site, an agent for improving the elastic properties of a vascular site, an antineoplastic substance, an anti-inflammatory substance, an antiplatelet substance, an anticoagulant substance, an antifibrin substance, an antithrombin substance, an antimitotic substance, an antibiotic substance, an antiallergy substance, an antioxidant substance, alpha-interferon, genetically engineered epithelial cells, rapamycin, or dexamethasone. In some of these embodiments, the device contains a polymeric layer coated on top of the ceramic component, on a portion of the device not containing the ceramic component, or the entire device. In some of these embodiments, attachment uses a laser as a heat source. In some of these embodiments, the ceramic component forms on heating a glass-frit slurry.

In another set of inventive embodiments, the medical device attaches to the ceramic component through an oxide layer formed on the surface of attachment regions machined into the medical device's surface. The ceramic component has a second less porous region at or near where it attaches to the oxide layer and a porous region substantially throughout the remainder. This porous region is filled with a drug at some time before use. In some of these embodiments, the surface of the medical device is a metal that comprises iron, cobalt, nickel, manganese, stainless steel, tantalum, niobium, super-elastic nickel-titanium alloys, titanium, silver, gold, platinum, steel, or aluminum. In some of these embodiments, the drug contains actinomycin D, or its derivatives and analog (manufactured by Sigma-Aldrich 1001 West Saint Paul Avenue, Milwaukee, Wis. 53233; or COSMEGEN available from Merck) including dactinomycin, actinomycin IV, actinomycin I₁, actinomycin X₁, and actinomycin C₁; paclitaxel (e.g. TAXOL® by Bristol-Myers Squibb Co., Stamford, Conn.); docetaxel (e.g. Taxotere®, from Aventis S.A., Frankfurt, Germany); methotrexate; azathioprine; vincristine; vinblastine; fluorouracil; doxorubicin hydrochloride (e.g. Adriamycin® from Pharmacia & Upjohn, Peapack N.J.); mitomycin (e.g. Mutamycin® from Bristol-Myers Squibb Co., Stamford, Conn.); sodium heparin; low-molecular-weight heparins; heparinoids; hirudin; argatroban; forskolin; vapiprost; prostacyclin and prostacyclin analogues; dextran; D-phe-pro-arg-chloromethylketone (synthetic antithrombin); dipyridamole; glycoprotein IIb/IIIa platelet membrane receptor antagonist antibody; recombinant hirudin; Angiomax™ (Biogen, Inc., Cambridge, Mass.); angiopeptin; angiotensin converting enzyme inhibitors; captopril (e.g. Capoten® and Capozide® from Bristol-Myers Squibb Co., Stamford, Conn.); cilazapril or lisinopril (e.g. Prinivil® and Prinzide® from Merck & Co., Inc., Whitehouse Station, N.J.); calcium channel blockers (such as nifedipine); colchicines; fibroblast growth factor (FGF) antagonists; fish oil (omega 3-fatty acid); histamine antagonists; lovastatin (an inhibitor of HMG-CoA reductase, a cholesterol lowering drug, brand name Mevacor® from Merck & Co., Inc., Whitehouse Station, N.J.); monoclonal antibodies (such as those specific for Platelet-Derived Growth Factor (PDGF) receptors); nitroprusside; phosphodiesterase inhibitors; prostaglandin inhibitors; suramin; serotonin blockers; steroids; thioprotease inhibitors; triazolopyrimidine (a PDGF antagonist); nitric oxide; rapamycin and its structual derivatives (Everolimus) and permirolast potassium. In some of these embodiments, the device contains a polymeric layer coated on top of the ceramic component, on a portion of the device not containing the ceramic component, or the entire device. In some of these embodiments, attachment uses a laser as a heat source.

In another set of embodiments, the ceramic component connects the surface of the medical device with the surface of an auxiliary component. Both of the surfaces attach to the ceramic component through an oxide layer on the surfaces (or on attachment regions machined or formed into the surfaces). The ceramic component has a second less porous region at or near where it attaches to each oxide layer. In some of these embodiments, the surface of the medical device is a metal that comprises iron, cobalt, nickel, manganese, stainless steel, tantalum, niobium, super-elastic nickel-titanium alloys, titanium, silver, gold, platinum, steel, or aluminum. In some of these embodiments, attachment uses a laser as a heat source. In some of these embodiments, the ceramic component forms on heating a glass-frit slurry.

In another set of embodiments, the ceramic component connects the surface of the medical device with the surface of an auxiliary component. Both of the surfaces attach to the ceramic component through an oxide layer on the surfaces (or on attachment regions machined or formed into the surfaces). The ceramic component has a second less porous region at or near where it attaches to each oxide layer. In some of these embodiments, the medical device is a stent. In some of these embodiments, the surface of the auxiliary component is a ceramic comprising titania, zirconia, hafnia, silica, alumina, silica alumina, silicon carbide, tungsten carbide, silicon boronitride, boronitride, silicon, or gallium arsenide. In some of these embodiments, attachment uses a laser as a heat source. In some of these embodiments, the ceramic component forms on heating a glass-frit slurry.

In another set of embodiments, the ceramic component connects the surface of the medical device with the surface of an auxiliary component. Both of the surfaces attach to the ceramic component through an oxide layer on the surfaces (or on attachment regions machined or formed into the surfaces). The ceramic component has a second less porous region at or near where it attaches to each oxide layer. In some of these embodiments, the surface of the medical device is a metal that comprises iron, cobalt, nickel, manganese, stainless steel, tantalum, niobium, super-elastic nickel-titanium alloys, titanium, silver, gold, platinum, steel, or aluminum. In some of these embodiments, the surface of the auxiliary component is a glass comprising borosilicate glass, lead glass, soda glass, uranium glass, soft glass, fused quartz, or fused silica. In some of these embodiments, the auxiliary component is a physical sensor. In some of these embodiments, attachment uses a laser as a heat source. In some of these embodiments, the ceramic component forms on heating the glass-frit slurry. In some of these embodiments, the medical device is a stent.

While particular embodiments of the present invention have been shown and described, it will be obvious to those skilled in the art that changes and modifications can be made without departing from this invention in its broader aspects. Therefore, the appended claims are to encompass within their scope all such changes and modifications as fall within the true spirit and scope of this invention.

1. A medical device for implanting in a patient comprising a ceramic component disposed on the surface of the medical device

2. The medical device of claim 1 wherein the ceramic component comprises a porous region.

3. The medical device of claim 2 further comprising at least one attachment region disposed within or on the surface wherein the ceramic component is disposed on or within the attachment region.

4. The medical device of claim 3 further comprising a second porous region wherein the second porous region is less porous than the first and wherein the ceramic component connects to at least one attachment region through the second porous region.

5. The medical device of claim 4 wherein the ceramic component is fused to the surface of at least one attachment region through the second porous region.

6. The medical device of claim 4 further comprising an oxide layer disposed between the surface of at least one attachment region and the second porous region.

7. The medical device of claim 1 wherein the porous region releasably contains a drug.

8. The medical device of claim 7 wherein the drug comprises at least one of a smooth-muscle-cell vascular activity inhibitor, a wound healing enhancer, an agent for improving the structural properties in a vascular site, an agent for improving the elastic properties of a vascular site, an antineoplastic substance, an anti-inflammatory substance, an antiplatelet substance, an anticoagulant substance, an antifibrin substance, an antithrombin substance, an antimitotic substance, an antibiotic substance, an antiallergy substance, an antioxidant substance, alpha-interferon, genetically engineered epithelial cells, rapamycin, actinomycin D, paclitaxel or docetaxel.

9. The medical device of claim 2 further comprising a polymer layer over the ceramic component, over a portion of the medical device not including the ceramic component, or both.

10. The medical device of claim 4 further comprising an auxiliary component with at least one auxiliary-component attachment region disposed in or on the surface of the auxiliary component and wherein the ceramic component is disposed on or within at least one auxiliary-component attachment region.

11. The medical device of claim 10 further comprising a third porous region disposed in the ceramic component wherein the third porous region is less porous than the first and wherein the ceramic component connects to at least one auxiliary-component attachment region through the third porous region.

12. The medical device of claim 11 wherein the ceramic component is fused to at least one auxiliary-component attachment region through the third porous region.

13. The medical device of claim 11 further comprising a second oxide layer disposed between the third porous region and at least one auxiliary-component attachment region.

14. The medical device of claim 11 wherein the surface or auxiliary-component surface, or both, comprise a metal, glass, or ceramic.

15. The medical device of claim 14 wherein metal comprises iron, cobalt, nickel, manganese, stainless steel, tantalum, niobium, super-elastic nickel-titanium alloys, titanium, silver, gold, platinum, steel, or aluminum.

16. The medical device of claim 14 wherein glass comprises borosilicate glass, lead glass, soda glass, uranium glass, soft glass, fused quartz, or fused silica.

17. The medical device of claim 14 wherein ceramic comprises carbide ceramics, oxide ceramics, nitride ceramics, or boride ceramics.

18. The medical device of claim 17 wherein ceramic comprises titania, zirconia, hafnia, silica, alumina, silica alumina, silicon carbide, tungsten carbide, silicon boronitride, boronitride, silicon, or gallium arsenide.

19. The medical device of claim 10 wherein the auxiliary component is one of an electrode, a physical sensor, or a chemical sensor.

20. The medical device of claim 10 further comprising a polymer layer disposed over the auxiliary component, over a portion of the medical device not including the auxiliary component, or both.

21. The medical device of claim 10 wherein the medical device is a stent.

22. The medical device of claim 1 wherein the surface of the medical device comprises plastic, metal, glass, or ceramic.

23. The medical device of claim 22 wherein metal comprises iron, cobalt, nickel, manganese, stainless steel, tantalum, niobium, super-elastic nickel-titanium alloys, titanium, silver, gold, platinum, steel, or aluminum.

24. The medical device of claim 22 wherein glass comprises borosilicate glass, lead glass, soda glass, uranium glass, soft glass, fused quartz, or fused silica.

25. The medical device of claim 22 wherein ceramic comprises carbide ceramics, oxide ceramics, nitride ceramics, or boride ceramics.

26. The medical device of claim 25 wherein ceramic comprises titania, zirconia, hafnia, silica, alumina, silica alumina, silicon carbide, tungsten carbide, silicon boronitride, boronitride, silicon, or gallium arsenide.

27. A medical device for implanting in a patient comprising:
a) a surface comprising a metal;
b) at least one attachment region disposed in or on the surface;
c) a ceramic component comprising a glass or ceramic, the ceramic component having a first porous region and a second less porous region, wherein the less porous region side of the ceramic component is fused on or within the attachment region; and
d) an oxide layer disposed on or within the attachment region between the surface of the device and the ceramic component.

28. The medical device of claim 27 wherein the medical device is a stent.

29. The medical device of claim 27 further comprising a drug releasably disposed in the first porous region.

30. A method of preparing a medical device comprising:
a) preparing at least one attachment region on or within the surface of a base medical device;
b) applying a ceramic-component precursor to the attachment region; and
c) converting the ceramic-component precursor into a ceramic component.

31. The method of claim 30 further comprising forming an oxide layer on or within at least one attachment region before the step of applying a ceramic-component precursor.

32. The method of claim 30 further comprising machining the base medical device, the surface of the base medical device, or a portion of the base medical device so that the base medical device, the surface of the base medical device, or a portion of the base medical device is thermally compatible with a ceramic component.

33. The method of claim 30 further comprising the step of fusing the ceramic component to the attachment region.

34. The method of claim 33 further comprising the step of fusing the ceramic component to the oxide layer.

35. The method of claim 30 wherein the step of preparing at least one attachment region in the surface uses a means for machining.

36. The method of claim 35 wherein the means for machining is laser ablation.

37. The method of claim 30 wherein the attachment region is prepared in the surface using a laser.

38. The method of claim 30 wherein converting the precursor into a ceramic component comprises heating the precursor and, optionally, at least a portion of the base medical device.

39. The method of claim 38 wherein the step of heating the precursor uses a means for heating.

40. The method of claim 39 wherein a means for heating is selected from furnaces, radiating heat sources, hydrogen furnaces, high-voltage DC arc current sources, or lasers.

41. The method of claim 38 wherein the ceramic component precursor is heated using a laser.

42. The method of claim 30 further comprising optionally preparing at least one attachment region on or within the surface of an auxiliary component and adhering the ceramic component into or onto the auxiliary-component attachment region.

43. The method of claim 42 further comprising forming an oxide layer on or within at least one auxiliary-component attachment region before the step of adhering the ceramic component.

44. The method of claim 30 wherein the ceramic component comprises a porous region.

45. The method of claim 44 wherein the porous region contains a drug.

46. The method of claim 45 further comprising the step of applying a polymer layer over the auxiliary component, over a portion of the base medical device not including the auxiliary component, or both.
